(19) United States
(12) Patent Application Publication
(10) Pub. No.: US 2004/0244806 A1
(43) Pub. Date: Dec. 9, 2004

(54) Treating disc herniation and other conditions with leukocytes

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/864,160

(22) Filed: Jun. 9, 2004

Related U.S. Application Data
(60) Provisional application No. 60/476,977, filed on Jun. 9, 2003.

(51) Int. Cl. A61F 2/44

(52) U.S. Cl. 128/898; 623/17.16

(57) ABSTRACT

Leukocytes, which may include one or more of monocytes, macrophages, lymphocytes, and neutrophils, are harvested and administered to a region of the body. In the preferred embodiment, leukocytes are harvested from the blood by plasmapherisis, a procedure known to those familiar with blood-banking techniques, and used to treat an intervertebral disc. Plasmapherisis allows the removal of desired blood component(s) from a larger volume of blood than the technique described in the '000 Application. Returning the non-desired blood components to the patient prior to withdraw of additional blood prevents complications that may occur from hypovolemia. Thus, a larger number of leukocytes can be obtained with plasmapherisis than with centrifugation of blood removed during a single blood withdraw. Plasmapherisis can also be used to harvest platelets to treat DDD, HNP and other non-disc-related conditions.

### REFERENCE TO RELATED APPLICATION

This application claims priority from U.S. Provisional Patent Application Ser. No. 60/476,977, filed Jun. 9, 2003, the entire content of which is incorporated herein by reference.

### FIELD OF THE INVENTION

This method relates generally to treatment of disc herniation or disc degeneration and, in particular, involves the treatment of disc herniation using leukocytes.

### BACKGROUND OF THE INVENTION

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

The human intervertebral disc is an oval to kidney bean shaped structure of variable size depending on the location in the spine. The outer portion of the disc is known as the annulus fibrosis. The annulus is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction).

The annulus contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

Interestingly, the adult disc is the largest avascular structure in the human body. Given the lack of vascularity, the nucleus is not exposed to the body's immune system. Most cells in the nucleus obtain their nutrition and fluid exchange through diffusion from small blood vessels in adjacent vertebra.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component.

Several hundred thousand patients undergo disc operations each year. Approximately five percent of these patients will suffer recurrent disc herniation, which results from a void or defect which remains in the outer layer (annulus fibrosis) of the disc after surgery involving partial discectomy. The defect acts as a pathway for additional material to protrude into the nerve, resulting in the recurrence of the herniation. This results in pain and further complications, in many cases.

Apart from destructive techniques, patients with herniated intervertebral discs and degenerative disc disease may conservatively be treated through rest, physical therapy, oral medication, and chiropractic care. Patients that do not respond to conservative care generally undergo an injection of steroids into the epidural space of their spinal canal (epidural space), or surgery. Steroid injection reduces the inflammation surrounding herniated or degenerated discs, which may reduce the pain from the disc. Unfortunately, steroid injection may hinder the healing process. Although growth factors and differentiation factors (soluble regulators) induce the healing process, it is believed that steroids may interfere with the cascade of these healing factors normally found in the body.

Given the large number of patients each year which require surgery for the treatment of disc disease and herniation, with substantial implications in terms of the cost of medical treatment and human suffering, any solution to improve the effectiveness of non-surgical treatments would be welcomed by the medical community.

### SUMMARY OF THE INVENTION

My U.S. patent application Ser. No. 09/897,000 (the '000 application) teaches, inter alia the treatment of Disc Herniation (HNP) and Degenerative Disc Disease (DDD) using concentrated growth and differentiation factors. This previously filed application also describes the harvest of Platelet Rich Plasma (PRP) from centrifugation of blood withdrawn from circulation.

The method described herein extends the techniques described in the '000 application. Broadly according to this invention, leukocytes, which may include monocytes, macrophages, lymphocytes, and neutrophils, are injected into a region of the body such as the spine to treat HNP and/or DDD. In the preferred embodiment, leukocytes are harvested from the blood by plasmapherisis, a procedure known to those familiar with blood-banking techniques. Plasmapherisis involves removing blood from a patient, extracting the desired blood component, then returning the remaining blood components that are not being harvested to the patient, and repeating the entire process.

Plasmapherisis allows the removal of desired blood component(s) from a larger volume of blood than the technique described in the '000 application. Returning the non-desired blood components to the patient prior to withdraw of additional blood prevents complications that may occur from hypovolemia. Thus, a larger number of leukocytes can be obtained with plasmapherisis than with centrifugation of blood removed during a single blood withdraw. Plasmapherisis can also be used to harvest platelets to treat DDD and HNP.

The inventions disclosed herein should offer a tremendous benefit to mankind. Healing injured avascular tissues, such as the intervertebral disc, will accelerate patients' recovery (decreased pain and quicker return to work), while helping patients avoid destructive surgical procedures.

### BRIEF DESCRIPTION OF THE DRAWINGS

FIG. 1 is an axial cross section of the spine, a lateral view of an endoscope, and an anterior view of three monitors;

FIG. 2 is an axial cross section of an HNP and a lateral view of an endoscope and a novel cutting instrument;

FIG. 3A is a lateral view of an embodiment of tip of the cutting instrument drawn in FIG. 2;

FIG. 3B is a lateral view of an alternative embodiment of the cutting instrument drawn in FIG. 3A;

FIG. 3C is a view of the distal end of the cutting instrument drawn in FIG. 3A;

FIG. 3D is a view of the proximal end of the cutting instrument drawn in FIG. 3B;

FIG. 3E is view of the top of the instrument drawn in FIG. 3A;

FIG. 3F is a view of the bottom of the instrument drawn in FIG. 3B;

FIG. 3G is a view of the bottom of the instrument drawn in FIG. 3A;

FIG. 3H is an axial view of a disc and a lateral view of the embodiment of the instrument drawn in FIG. 3B;

FIG. 4A is an axial cross section of a novel clip;

FIG. 4B is a lateral view of an endoscope, the clip drawn in FIG. 4A, and the knife drawn in FIG. 3B;

FIG. 4C is an axial cross section of the spine and the tip of an endoscope;

FIG. 5A is a lateral view of the tip of an instrument used to abrade the HNP; and

FIG. 5B is a view of the bottom of the tip of the instrument drawn in FIG. 5A.

### DETAILED DESCRIPTION OF THE INVENTION

As described in U.S. patent application Ser. No. 09/897,000, HNP is currently treated by surgical excision, physical therapy, chiropractic care, and medication. Frequently HNP is treated with steroids, which can be given orally. Alternatively, steroids can be injected into the spinal canal through a procedure know as epidural steroid injection. Steroids reduce inflammation. Reducing inflammation often decreases patient's pain. Inflammation is an important part of tissue healing. Thus, reducing patient's inflammation, and thus reducing tissue healing, may increase the likelihood a patient will require surgery to remove an HNP. Numerous studies have show steroids interfere with tissue healing.

The growth factors delivered with PRP, including TGF-beta and PDGF, recruit leukocytes to the area of application. This process, know as chemotaxis, attracts leukocytes from the blood to the area of application. The leukocytes, especially monocytes and macrophages are extremely important in tissue healing. Leukocytes release additional important growth factors including beta-FGF, VEGF, PDGF, TGF-beta1, Interlukin-1, and TNF-alpha. The released growth factors promote angiogenesis (new blood vessel formation), and tissue debridement. Leukocytes removed damaged extracellular matrix (ECM) and injured or dead cells. Beta-FGF released from macrophages leads to increased wound tensile strength and wound maturation.

The PRP described in the '000 application delivers leukocytes and chemotaxic factors that recruit additional leukocytes. The method taught in this application delivers a higher number of leukocytes. Furthermore, the method taught in the present application provides a fluid with a higher concentration of leukocytes. The method described in this application anticipates improvement in technology to provide fluids, pastes, or gels with higher leukocyte concentrations than that allowed by current equipment.

The method could be used in many other applications. The method could be used to: a) accelerate healing following surgically treated tissues including soft tissues, organs, bone, etc. b) promote bone healing in spinal fusion, fractures, pseudoarthrosis, and prosthetic components designed for bone ingrowth, etc. c) treat inflammatory disorders such as bursitis, tendonitis, including tennis elbow, and rotator cuff tendonitis or bursitis d) infections including discitis, osteomyelitis, pelvic inflammatory disease, diverticulitis, etc. e) arthritis f) sprains and strains g) other medical conditions.

Plasmapherisis could also be used to obtain other fractions of the blood to treat the diseases and conditions mentioned above. For example, plasmapherisis could be used to obtain platelets. More than one blood fraction could be harvested via plasmapherisis. The components could be combined and applied to the area of interest. For example, leukocytes and platelets harvested via plasmapherisis could be combined and applied to a surgical site. Growth factors, proteases, and other cytokines, could be added to the blood fraction harvested via plasmapherisis. The added factors or cells could be obtained through recombinant technology or cell culture. To reduce pain, topical anesthetic agents may be applied with the component obtained via plasmapherisis.

Tissue healing is regulated by a tightly controlled, complex, cascade of growth factors, cytokines, and proteases. The regulatory proteins control migration and mitosis (reproduction) of cells, debridement of damaged extracellular matrix (ECM), removal of damaged and dead cells, regeneration of the ECM, and remodeling of the ECM.

The healing of damaged tissues can be divided into three overlapping phases: a) inflammatory phase, b) repair phase, and c) remodeling phase.

The inflammatory phase is initiated by injured blood vessels within the damaged tissue. Blood vessel injury leads to extravasation of blood and blood clots. Clotting of blood occurs as platelets adhere to one another and to the surrounding tissues. Platelets release their alpha-granules during the clotting process. The alpha-granules contain growth factors including Platelet Derived Growth Factor (PDGF), Insulin-like Growth Factors (IGF), Epidermal Growth Factor (EGF), and Transforming Growth Factors-Beta (TGF-beta).

The platelet released factors encourage migration of inflammatory cells to the damaged tissues through a process known as chemotaxis. Neutrophils are the first inflammatory cells attracted to the injured tissues. Polymorphonuclear leukocytes (PMNs) account for 95% of the cells in the area surrounding the blood clot two days after injury.

Neutrophils secrete inflammatory cytokines into the damaged tissues. Neutrophils also engulf and destroy bacteria. PMNs release proteases. Proteases, including Matrix Metalloproteinases (MMPs), elastase, and collagenase, debride the damaged tissues.

Transforming Growth Factor-Beta attracts monocytes to the damaged tissue. Products released from the action of the proteases on the ECM also attract monocytes. For example, fragments of fibronectin attract monocytes. Monocytes transform into activated macrophages. Macrophages replace PMNs as the primary inflammatory cells in the damaged tissue. Macrophages account for 50-80% of the inflammatory cells in the injured tissues six days after injury.

Macrophages remove damaged cells, dead cells, and damaged ECM. Macrophages also release proinflammatory cytokines and growth factors including Tissue Necrosis Factor-alpha (TNF-alpha), Interleukin-1 (IL-1), TGF-beta, TGF-alpha, Leukocyte Derived Growth Factor (LDGF), Basic Fibroblastic Growth Factor (bFGF), Vascular Endothelial Growth Factor (VEGF), and Heparin-Binding Epidermal Growth Factor (HB-EGF). Macrophages, and the cells stimulated by macrophages, release proteases including urokinase, plasminogen activators, and collagenase. The proteases assist with debridement.

The repair phase is orchestrated by macrophage recruited fibroblasts, epithelial cells, and vascular endothelial cells. The repair phase is characterized by angiogenesis (new blood vessel formation), and ECM synthesis; The cells of the repair phase also secrete growth factors. Fibroblasts release IGF-1, bFGF, TGF-beta, PDGF, and Keratinocyte Growth Factor (KGF). Endothelial cells release bFGF and PDGF. Keratinocytes produce TGF-beta and TGF-alpha. The released factors stimulate mitosis, angiogenesis, and ECM synthesis.

The remodeling phase is largely controlled by fibroblasts. The remodeling phase lasts for months. During this phase, the scar matrix matures through carefully coordinated ECM synthesis and debridement.

The intervertebral disc is the largest avascular structure in the human body. The paucity of blood vessels hinders the healing process. Unless a herniated disc tears a blood vessel, there is little chance the body will invoke a vigorous healing response. The healing of injured tissues begins with vessel injury, platelet degranulation, and leukocyte infiltration. The avascular disc, as well as other relatively avascular tissues of the body (tendons, ligaments, cartilage, and fibrocartilage), have poor healing characteristics. The preferred embodiment of my invention adds a blood clot, leukocytes, and concentrated growth factors to the injured (HNP), avascular, disc. Other embodiments of the invention add growth factors alone. The growth factors may be used in combination. Growth factors may act additively or synergistically with each other and other proteins.

Surgery for Herniated discs (HNP) requires removal of the disc fragment that impinges upon the nerve or spinal cord. Unfortunately, the surgery also involves removal of a generous portion of the Nucleus Pulposus contained within the disc. Nucleus Pulposus removal accelerates disc degeneration. Many patients suffer continued back and neck pain following disc surgery. As many as 30 percent of patients undergoing disc surgery will require additional spine surgeries.

Epidural Steroid Injections (ESI) are currently used to treat HNP ESI decrease inflammation surrounding the HNP. Decreased inflammation may decrease a patient's pain. Leukocyte infiltration and inflammation are necessary for tissue healing. As noted previously, the lack of blood vessels within the disc compromises the disc's ability to heal. Decreasing inflammation with steroids, oral or injected, further compromise disc healing. Steroids impede healing of injured soft tissues and injured bones. Steroids decrease PMN infiltration, decrease fibroblast replication, decrease collagen synthesis, decrease wound macrophage numbers, and decrease wound TGF-beta concentrations. Pierce et al. showed that a dose of 30 mg of methyleprednislone per Kg of body weight decreased wound breaking strength by 75 percent at five days after the administration of the steroid. The authors also noted the steroid decreased the number of wound macrophages to near undetectable levels. Pierce et al. state that the influx of macrophages into the wound the first few days after wounding is an absolute requirement for normal healing. Thus, ESI, the current “gold standard” in the treatment of HNP, likely increase the probability patients will require surgery by impeding the body's ability to resorb the HNP.

Most of the cytokines, or growth factors, may be obtained from pharmaceutical companies that supply proteins that are manufactured with recombinant technology. Recombinant growth hormone (rhGH) (Genetech, San Francisco, Calif.), Vascular endothelial growth factor (rhVEGF) (Genetech, San Francisco, Calif.), Platelet derived growth factor (rhPDGF-AA & rhPDGF-BB) (Chiron, Emeryville, Calif. & Amgen, Thousand Oaks, Calif.), Transforming growth factor-beta (rhTGF-Beta) (Oncogen Corp, Seattle, Wash., New England Nuclear Boston, Mass., R&D Systems, Minneapolis, Minn., & Amgen, Thousand Oaks, Calif.), Fibroblastic growth factor (rhaFGF & rhbFGF) (Amgen, Thousand Oaks, Calif. & Chiron, Emeryville, Calif.), Insulin-like growth factor (rhIGF-1) (UBI Lake Placid, N.Y. & Amgen, Thousand Oaks, Calif.), Granulocyte colony stimulating factor (rhGM-CSF, rhG-CSF) (Amgen, Thousand Oaks, Calif.), Macrophage colony stimulating factor (rhM-CSF) (Chiron, Emeryville, Calif.), and Epidermal growth factor (rhEGF) (Chiron, Emeryville, Calif.) have been used to stimulate healing of chronic cutaneous ulcers. Several of the cytokines have also been used to stimulate healing of other skin wounds including burns, skin graft donor sites, and incisions in the skin. The factors have been applied to the skin and they have been administered systemically via injection. The FDA has approved rhPDGF for the treatment of diabetic skin ulcers.

The cytokines have been less effective in promoting the healing of cutaneous ulcers than researchers hoped. It is thought the diminished blood supply, bacterial contamination of the wound, and the elderly age of the patients adversely affected the results of the skin ulcer studies.

Application of cytokines and leukocytes to HNPs will likely benefit patients more than the application of cytokines to cutaneous ulcers. First, patients with HNPs are generally younger than patients with diabetic or vascular ulcers. Cytokines have been shown to work better in younger patients. Second, the area around the HNP is sterile. Bacterial contamination of open wounds leads to the release of proteases. Proteases denature the cytokines. Third, unlike the compromised blood supply of the tissues that surround cutaneous ulcers, the epideral space has abundant blood vessels.

This invention increases the number of macrophages at the site of the HNP in number of ways. The macrophages may be concentrated from a patient's blood and injected into the epideral space around the HNP. The macrophages may also be injected into the disc fragment or the disc. Macrophages accelerate the resorption of the disc fragment and speed the healing of the Annulus Fibrosus (AF). In fact, the novel methods taught in this application may be used to treat tears of the AF, without concominant HNP.

Leukocytes may be obtained from patients through a process known as Leukapheresis. Apheresis is a well know blood banking technique. Leukocytes may be obtained from the buffy coat of centrifuged blood. Highly purified populations of monocytes may be isolated by counterflow centrifugal elutriation of heparinized blood of patients undergoing Leukapheresis. The macrophages may be activated prior to injecting them into the patient. Methods of activating macrophages are taught in the text “The Macrophage” by Burke et al.

The number of circulating leukocytes may also be increased by local or systemic administration of rhM-CSF, rhGM-CSF, rhG-CSF or other related factor. Increased number circulating leukocytes leads to increased number of macrophages in the area surrounding the HNP. The factors upregulate macrophage adhesion, mitotic activity, and differentiation. For example, 2.5 to 100 ug per Kg of body weight of one of the above mentioned factors could be injected subcutaneously. The dose could be repeated daily for seven days. Alternatively 400 ug could be injected into the epideral space around the HNP.

Cytokines may also be used as chemotactic factors to attract macrophages to the HNP, as taught in my co-pending U.S. patent application Ser. No. 09/897,000. The factors may be injected into the epideral space, into the disc fragment, into the disc, or into the area about a tear in the AF. The factors may be administered in a single dose. Alternatively, the factors may be administered in a series of injections over several days or weeks. A single factor may be injected. Alternatively, the factors may be combined with each other and/or leukocytes. For example, rhVEGF (500-100 ug, or 1-3 mg/kg), rhGH (0.2 mg/kg/day for seven days), rhPDGF-BB (100-600 ug per day for weeks), rhTGF-Beta (2-600 ug per day for 5 days), rhbFGF (100 ug to 5 mgs per day for six days), rhEGF (40 ug per day for three days) or a combination of two or more of the cytokines could be used.

The listed dosages are examples; the invention anticipates modifying the recommended dosages after gaining further experience with the cytokines. For example, rhBMP-2, a TFG-Beta superfamily cytokine, is used to grow bone. The recommended dosages for rhBMP-2 have increased 40 fold over the last two decades. The recommended dosages of the cytokines listed above will likely increase. In fact the recommended dosages of the cytokines will likely increase to 10-40 mg per treatment. The recommended dosage of rhBMP-2 to grow bone has increased to 24 mg per treatment. The invention also anticipates the use of additional cytokines as the new cytokines become available through recombinant techniques.

Additional factors may be injected with or without the above mentioned factors. For example, TNF-alpha, MIP-1apha, Glucan, interferon, prostaglandin synthases inhibitors, heparin-binding fragments of fibronectin, protamine, and angiostatic steroids could be used to attract macrophages to the HNP.

The cytokines may be incorporated into carriers as taught in my U.S. Pat. Nos. 6,340,369 and 6,454,804. Carriers allow sustained release of the cytokines. Carriers may also protect the cytokines from proteases. The preferred carriers are resorbable. The carriers may include collagen, polymers, hydrogels, or related materials. For example, the carriers could include Carboxymethylcellulose gel, (Scios, Mountainview, Calif.), encapsulation of cytokine-gelatin particles with oligopoly(ethylene glycol)fumarate (OPF), emulsified collagen (Zyderm II, Collagen Corp., Palo Alto, Calif.), recombinant human collagen (Collagen Corp., Palo Alto, Calif.).

The '000 application describes the harvest of platelets and leukocytes to treat disc Herniation and Degenerative Disc Disease; it also describes the use of calcium and thrombin to force the platelets to degranulate. Degranulating platelets release latent TGF-B. My co-pending application U.S. Ser. No. 60/519,397 teaches the use of proteases to activate the latent TGF-B. The proteases are added to the Platelet Rich Plasma (PRP), calcium, and thrombin. The proteases include plasmin, calpin, MMP-9, thrombospondin, transglutaminase, the mannose 6-phosphate receptor (M6PR), furin, substilisin-like endoproteases, and integrins.

Dermatopontin can also be added to the PRP to enhance its biologic activity.

### EXAMPLE

A preferred embodiment of this invention has been used to treat five patients with HNPs. All five patients were referred for surgical removal of their disc, at the failure of conservative, prior-art treatments. PRP was harvested from an aspirate of blood as described in the pending patent. Three ccs of the PRP, calcium, and thrombin mixture was injected into the epidural space via a transforaminal epidural injection. The patients were followed clinically by the anesthesiologist who performed the injections.

All patients experienced dramatic improvement of their symptoms. The patients noted decreased back pain, decreased leg pain, improved strength of their legs, and improved sensation over their legs. Four of the patients reported enough improvement with a single injection of PRP that they no longer desired to undergo spinal surgery. MRI scans two months following PRP injection showed at least partial resorption of the HNP in four patients.

The cytokines could also be delivered to the area of the HNP from cells infected with the viruses encoded with the genetic material that guides the cells to produce the cytokines. Genetic engineering techniques are described by Chandler et al.

Additional therapeutic treatments used to assist the cytokine and leukocytes. For example, the disc or disc fragment may be abraded or incised to ease the migration of macrophages into the fragment. Abraidment increases the HNP surface area and removes tissues that may impede the migration of macrophages. Incision or abraidment of the HNP may be performed endoscopically. CT or MRI guidance may be used with or without endoscopy. The endoscope and the instruments used with the endoscope may be electrified as described in my co-pending application U.S. patent application Ser. No. 10/842,192. The novel techniques taught in the application helps surgeons avoid injuring spinal nerves.

At least a few of the cells within the disc fragment could be killed prior to application of the cytokines. An enzyme or toxin, such as chymopapain or chondrointinase ABC could be injected into the disc fragment. An electric current could be applied to the fragment. For example, bipolar electric cautery could be used to kill some of the cells in the HNP. Other techniques including radio-frequency, ultrasonic desiccation, laser surgery, and cryosurgery could be used to kill at least some of the cells in the HNP.

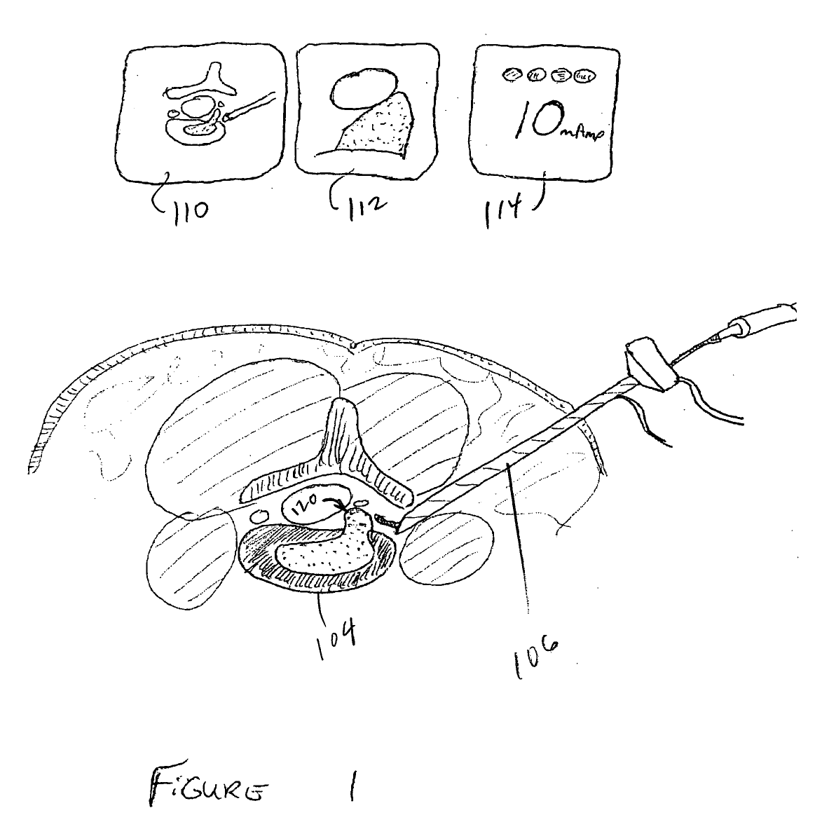

FIG. 1 is an axial cross section of the spine, a lateral view of an endoscope, and an anterior view of three monitors. A HNP is depicted at 102, and the annulus fibrosis (AF) is shown at 104. An endoscope 106 is seen coursing through the skin and muscles. The endoscope was directed to the HNP. A catheter extending through a channel in the endoscope is used to inject the cytokines and the carrier over the HNP. The monitor 110 represents a CT or MRI monitor. The CT or MRI monitor shows the spine and the endoscope. The monitor 112 represents the view provided by the endoscope. The endoscope monitor shows an enlarged view of the HNP and a nerve compressed by the HNP. The monitor 114 represents a Neurophysiology (NP) monitor, which records the electrical activity of the nerves and or the muscles. The (NP) monitor 114 alerts the surgeon if the instruments are applying too much pressure to the nerves. The (NP) monitor could record spontaneous EMG activity or electrically stimulated activity.

FIG. 2 is an axial cross section of an HNP and a lateral view of an endoscope and a novel cutting instrument 210. The instrument is an embodiment of the instrument described in my co-pending U.S. Patent Application Ser. No. 60/479,718 the entire content of which is incorporated herein by reference. The instrument has a blunt edge that may be used to dissect between the nerves and the HNP. The instrument has a sharp cutting edge below the blunt edge.

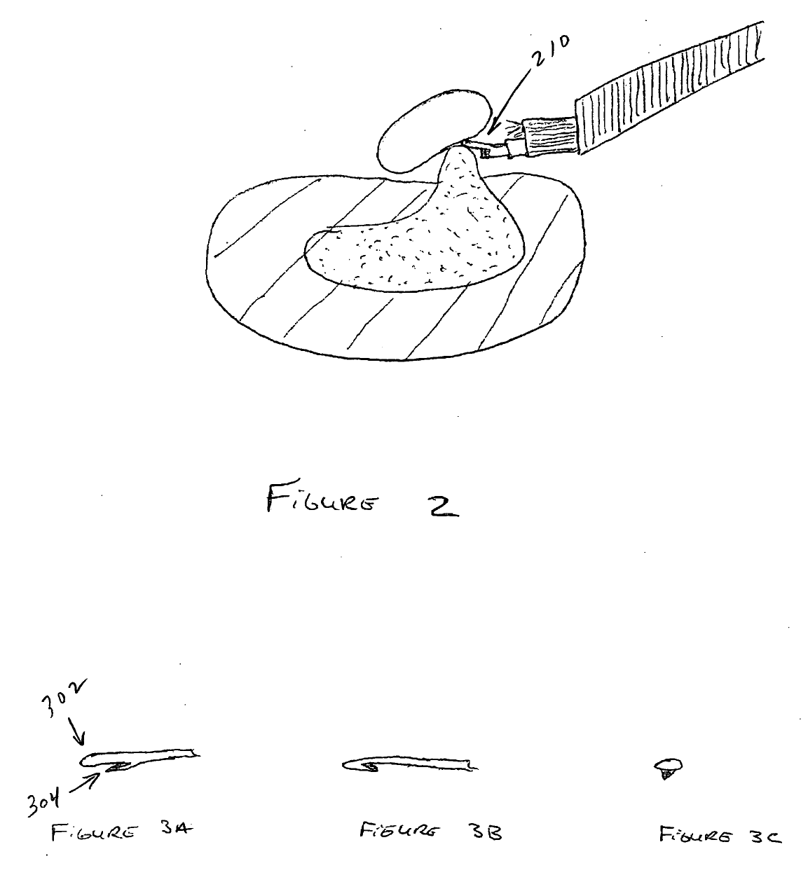

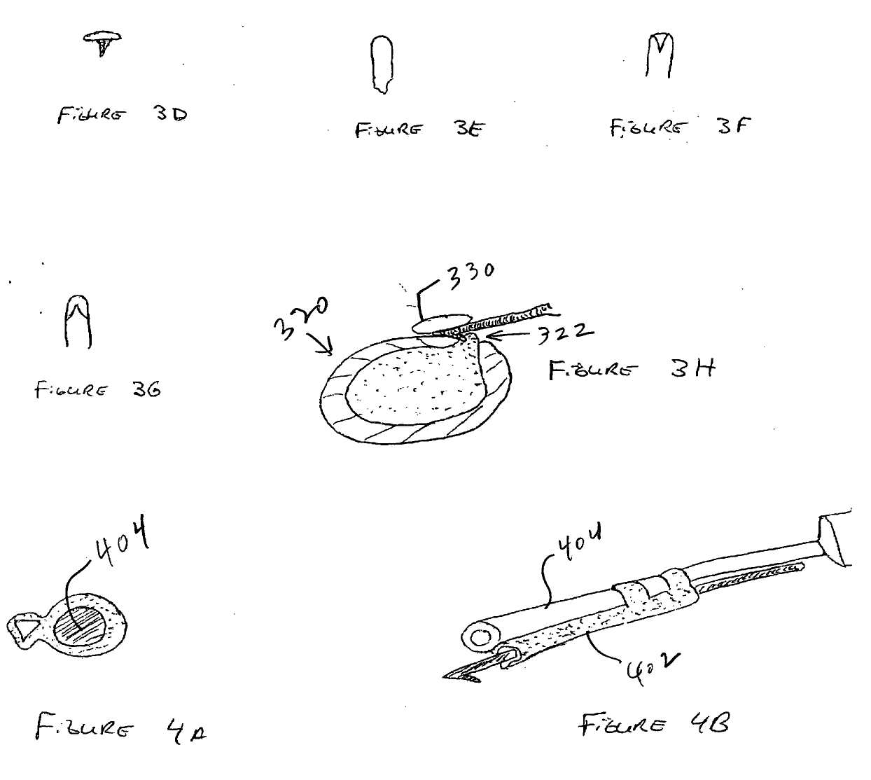

FIG. 3A is a lateral view of an embodiment of tip of the cutting instrument drawn in FIG. 2. The blunt edge 302 is located on the superior surface of the instrument, whereas the cutting edge 304 is located on the inferior surface of the instrument. The instrument cuts by pushing the tool. FIG. 3B is a lateral view of an alternative embodiment of the cutting instrument drawn in FIG. 3A. The instrument cuts by pulling the instrument. FIG. 3C is a view of the distal end of the cutting instrument drawn in FIG. 3A. FIG. 3D is a view of the proximal end of the cutting instrument drawn in FIG. 3B. FIG. 3E is view of the top of the instrument drawn in FIG. 3A. FIG. 3F is a view of the bottom of the instrument drawn in FIG. 3B. FIG. 3G is a view of the bottom of the instrument drawn in FIG. 3A. FIG. 3H is an axial view of a disc 320 and a lateral view of the embodiment of the instrument drawn in FIG. 3B. The blunt edge of the knife lies between the HNP 322 and the nerve 330. The instrument cuts the HNP as it is pulled towards the surgeon.

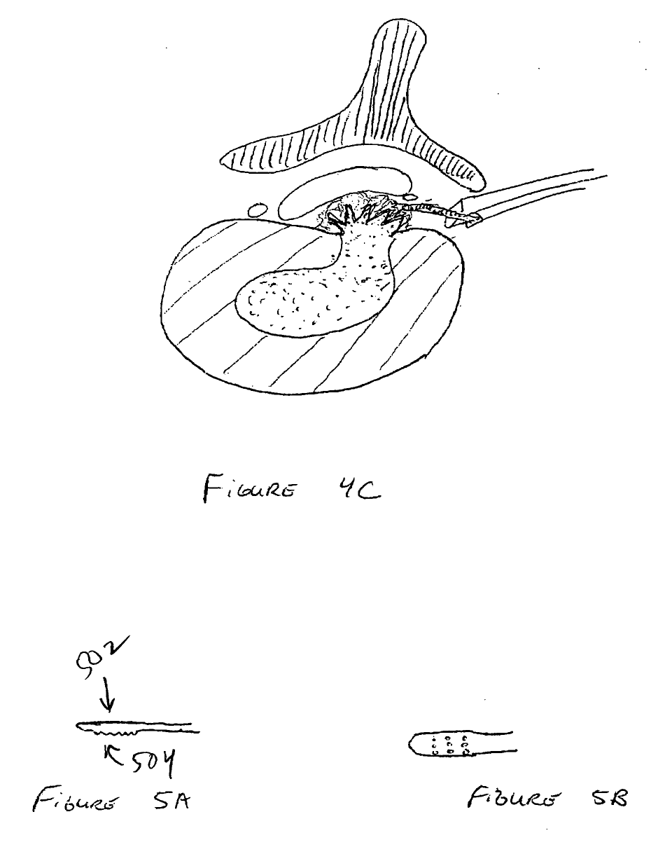

FIG. 4A is an axial cross section of a novel clip 402 that may attach to an endoscope 404. The area of the drawing with the diagonal lines represents the endoscope. The lumen of the clip is shaped to only permit the knife of FIG. 3A to inserted with the blunt tip towards the nerves. FIG. 4B is a lateral view of an endoscope, the clip drawn in FIG. 4A, and the knife drawn in FIG. 3B. FIG. 4C is an axial cross section of the spine and the tip of an endoscope. The HNP has been cut by the knife drawn in FIG. 4B. A catheter is drawn applying the cytokines over the freshly cut surface of the HNP.

FIG. 5A is a lateral view of the tip of an instrument used to abrade the HNP. The instrument's upper surface 502 is blunt. The instrument's lower surface 504 has multiple projections. FIG. 5B is a view of the bottom of the tip of the instrument drawn in FIG. 5A.

I claim:

1. A method of treating a medical condition, comprising the steps of:
harvesting leukocytes from blood; and
administering the harvested leukocytes to a region of the body.

2. The method of claim 1, wherein the leukocytes are harvested from the blood using plasmapherisis.

3. The method of claim 1, wherein the leukocytes are injected into the spine to treat HNP.

4. The method of claim 1, wherein the leukocytes include one or more of the following:
monocytes, macrophages, lymphocytes, and neutrophils.
